(19) United States
(12) Patent Application Publication
(10) Pub. No.: US 2002/0127662 A1
(43) Pub. Date: Sep. 12, 2002

(54) Process for the fermentative preparation of L-glutamic acid using coryneform bacteria

(76) Inventors: Lothar Eggeling, Julich (DE); Karin Krumbach, Duren (DE); Hermann Sahm, Julich (DE); Georg Thierbach, Bielefeld (DE)

Correspondence Address: SMITH, GAMBRELL & RUSSELL, LLP, SUITE 800, 1850 M STREET, N.W., WASHINGTON, DC, 20036, US

(21) Appl. No.: 09/909,850

(22) Filed: Jul. 23, 2001

Related U.S. Application Data
(60) Provisional application No. 60/220,395, filed on Jul. 24, 2000.
(60) Provisional application No. 60/292,514, filed on May 23, 2001.

(51) Int. Cl. C12P 13/14; C12N 9/02; C12N 1/21

(52) U.S. Cl. 435/110; 435/252.3; 435/189

(57) ABSTRACT

The invention relates to a process for the preparation of L-glutamic acid by fermentation of coryneform bacteria, in which bacteria in which the nucleotide sequence which codes for D-alanine racemase (alr gene) is attenuated are employed, wherein the following steps are carried out:

a) fermentation of the L-glutamic acid-producing bacteria in which at least the gene which codes for D-alanine racemase is attenuated,

b) concentration of the L-glutamic acid in the medium or in the cells of the bacteria and

c) isolation of the L-glutamic acid produced.

### CROSS-REFERENCE TO RELATED APPLICATIONS

This application claims priority to U.S. Provisional Application No. 60/220,395, filed on Jul. 24, 2000 and to U.S. Provisional Application No. 60/292,514, filed on May 23, 2001. Both provisional applications are hereby incorporated by reference in their entirety.

### BACKGROUND OF THE INVENTION

The invention relates to a process for the fermentative preparation of L-glutamate using coryneform bacteria in which the alr gene is attenuated. All references cited herein are expressly incorporated by reference. Incorporation by reference is also designated by the term “I.B.R.” following any citation.

Amino acids, such as L-glutamate, are used in human medicine and in the pharmaceuticals industry and in the foodstuffs industry.

### BRIEF SUMMARY OF THE INVENTION

It is known that amino acids are prepared by fermentation from strains of coryneform bacteria, in particular Corynebacterium glutamicum. Because of their great importance, work is constantly being undertaken to improve the preparation processes. Improvements to the process can relate to fermentation measures, such as e.g. stirring and supply of oxygen, or the composition of the nutrient media, such as e.g. the sugar concentration during the fermentation, or the working up to the product form by e.g. ion exchange chromatography, or the intrinsic output properties of the microorganism itself.

Methods of mutagenesis, selection and mutant selection are used to improve the output properties of these microorganisms. Strains which are resistant to antimetabolites or are auxotrophic for metabolites of regulatory importance and produce L-amino acids are obtained in this manner.

Methods of the recombinant DNA technique have also been employed for some years for improving the strain of Corynebacterium strains which produce L-amino acid.

The invention provides new measures for improved fermentative preparation of L-glutamate.

### BRIEF DESCRIPTION OF THE FIGURE

FIG. 1: Map of the plasmid pK18mobalr

The base pair numbers stated are approximate values obtained in the context of reproducibility of measurements. The abbreviations and designations used have the following meaning:

alr: alr (alanine racemase) gene from C. glutamicum ATCC13032

Kan: Kanamycin resistance gene

oriT: Origin of transfer

ApoI: Cleavage site of the restriction enzyme ApoI

SacI: Cleavage site of the restriction enzyme SacI

SphI: Cleavage site of the restriction enzyme SphI

HincII: Cleavage site of the restriction enzyme HincII

HindIII: Cleavage site of the restriction enzyme HindIII

PvuI: Cleavage site of the restriction enzyme PvuI

BglI: Cleavage site of the restriction enzyme BglI

BglII: Cleavage site of the restriction enzyme BglII

BclI: Cleavage site of the restriction enzyme BclI

### DETAILED DESCRIPTION OF THE INVENTION

When L-glutamic acid or glutamic acid or L-glutamate or glutamate are mentioned in the following text, this means not only the free acids but also the salts of L-glutamic acid, such as e.g. the calcium, sodium, ammonium or potassium salt.

The invention provides a process for the fermentative preparation of L-glutamic acid using coryneform bacteria in which at least the nucleotide sequence which codes for the enzyme D-alanine racemase (alr gene) is attenuated.

The strains employed preferably already produce L-glutamic acid before attenuation of the alr gene.

The term “attenuation” or “attenuate” in this connection describes the reduction or elimination of the intracellular activity of one or more enzymes (proteins) in a microorganism which are coded by the corresponding DNA, for example by using a weak promoter or a gene or allele which codes for a corresponding enzyme with a low activity or inactivates the corresponding enzyme (protein), and optionally combining these measures.

The microorganisms which the present invention provides can produce L-glutamic acid from glucose, sucrose, lactose, fructose, maltose, molasses, starch, cellulose or from glycerol and ethanol. They are representatives of coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular of the species Corynebacterium glutamicum, are, for example, the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Brevibacterium flavum ATCC14067

Brevibacterium lactofermentum ATCC13869 and

Brevibacterium divaricatum ATCC14020

and mutants or strains prepared therefrom which over-produce L-glutamic acid.

It has been found that coryneform bacteria produce L-glutamic acid in an improved manner after attenuation of the alr gene which codes for D-alanine racemase.

The nucleotide sequence of the alr gene is shown in SEQ ID No 1 and the enzyme protein amino acid sequence resulting therefrom is shown in SEQ ID No 2.

The alr gene described in SEQ ID No 1 is employed according to the invention. Alleles of the alr gene which, for example, result from the degeneracy of the genetic code or due to sense mutations of neutral function can furthermore be used.

To achieve an attenuation, either the expression of the alr gene or the catalytic properties of the enzyme protein can be reduced or eliminated. The two measures can optionally be combined.

The reduction in gene expression can take place by suitable culturing or by genetic modification (mutation) of the signal structures of gene expression. Signal structures of gene expression are, for example, repressor genes, activator genes, operators, promoters, attenuators, ribosome binding sites, the start codon and terminators. The expert can find information on this e.g. in the patent application WO 96/15246 I.B.R., in Boyd and Murphy (Journal of Bacteriology 170: 5949 (1988)) I.B.R., in Voskuil and Chambliss (Nucleic Acids Research 26: 3548 (1998) I.B.R., in Jensen and Hammer (Biotechnology and Bioengineering 58: 191 (1998)) I.B.R., in Patek et al. (Microbiology 142: 1297 (1996) I.B.R. and in known textbooks of genetics and molecular biology, such as e. g. the textbook by Knippers (“Molekulare Genetik [Molecular Genetics]”, 6th edition, Georg Thieme Verlag, Stuttgart, Germany, 1995) I.B.R. or that by Winnacker (“Gene und Klone [Genes and Clones]”, VCH Verlagsgesellschaft, Weinheim, Germany, 1990) I.B.R.

Mutations which lead to a change or reduction in the catalytic properties of enzyme proteins are known from the prior art; examples which may be mentioned are the works by Qiu and Goodman (Journal of Biological Chemistry 272: 8611-8617 (1997)) I.B.R., Sugimoto et al. (Bioscience Biotechnology and Biochemistry 61: 1760-1762 (1997)) I.B.R. and Möckel (“Die Threonindehydratase aus Corynebacterium glutamicum: Aufhebung der allosterischen Regulation und Struktur des Enzyms [Threonine dehydratase from Corynebacterium glutamicum: Cancelling the allosteric regulation and structure of the enzyme]”, Reports from the Jülich Research Centre, Jül-2906, ISSN09442952, Jülich, Germany, 1994) I.B.R. Comprehensive descriptions can be found in known textbooks of genetics and molecular biology, such as e. g. that by Hagemann (“Allgemeine Genetik [General Genetics]”, Gustav Fischer Verlag, Stuttgart, 1986) I.B.R.

Possible mutations are transitions, transversions, insertions and deletions. Depending on the effect of the amino acid exchange on the enzyme activity, missense mutations or nonsense mutations are referred to. Insertions or deletions of at least one base pair in a gene lead to frame shift mutations, which lead to incorrect amino acids being incorporated or translation being interrupted prematurely. Deletions of several codons typically lead to a complete loss of the enzyme activity. Instructions on generation of such mutations are prior art and can be found in known textbooks of genetics and molecular biology, such as e. g. the textbook by Knippers (“Molekulare Genetik [Molecular Genetics]”, 6th edition, Georg Thieme Verlag, Stuttgart, Germany, 1995) I.B.R., that bv Winnacker (“Gene und Klone [Genes and Clones]”, VCH Verlagsgesellschaft, Weinheim, Germany, 1990) I.B.R. or that by Hagemann (“Allgemeine Genetik [General Genetics]”, Gustav Fischer Verlag, Stuttgart, 1986) I.B.R.

Another common method of mutating genes of C. glutamicum is the method of gene disruption and gene replacement described by Schwarzer and Pühler (Bio/Technology 9, 84-87 (1991)) I.B.R.

In the method of gene disruption a central part of the coding region of the gene of interest is cloned in a plasmid vector which can replicate in a host (typically E. coli), but not in C. glutamicum. Possible vectors are, for example, pSUP301 (Simon et al., Bio/Technology 1, 784-791 (1983)) I.B.R., pK18mob or pK19mob (Schäfer et al., Gene 145, 69-73 (1994)) I.B.R., pK18mobsacB or pK19mobsacB (Jäger et al., Journal of Bacteriology 174: 5462-65 (1992) I.B.R.), pGEM-T (Promega corporation, Madison, Wis., USA), pCR2.1-TOPO (Shuman (1994). Journal of Biological Chemistry 269:32678-84 I.B.R.; U.S. Pat. No. 5,487,993 I.B.R.), pCR®Blunt (Invitrogen, Groningen, Holland; Bernard et al., Journal of Molecular Biology, 234: 534-541 (1993) I.B.R.) or pEM1 (Schrumpf et al, 1991, Journal of Bacteriology 173:4510-4516) I.B.R. The plasmid vector which contains the central part of the coding region of the gene is then transferred into the desired strain of C. glutamicum by conjugation or transformation. The method of conjugation is described, for example, by Schäfer et al. (Applied and Environmental Microbiology 60, 756-759 (1994)) I.B.R. Methods for transformation are described, for example, by Thierbach et al. (Applied Microbiology and Biotechnology 29, 356-362 (1988)) I.B.R., Dunican and Shivnan (Bio/Technology 7, 1067-1070 (1989)) I.B.R. and Tauch et al. (FEMS Microbiological Letters 123, 343-347 (1994)) I.B.R. After homologous recombination by means of a “cross over” event, the coding region of the gene in question is interrupted by the vector sequence and two incomplete alleles are obtained, one lacking the 3′ end and one lacking the 5′ end. This method has been used, for example, by Fitzpatrick et al. (Applied Microbiology and Biotechnology 42, 575-580 (1994)) I.B.R. to eliminate the recA gene of C. glutamicum.

In the method of gene replacement, a mutation, such as e.g. a deletion, insertion or base exchange, is established in vitro in the gene of interest. The allele prepared is in turn cloned in a vector which is not replicative for C. glutamicum and this is then transferred into the desired host of C. glutamicum by transformation or conjugation. After homologous recombination by means of a first “cross-over” event which effects integration and a suitable second “cross-over” event which effects excision in the target gene or in the target sequence, the incorporation of the mutation or of the allele is achieved. This method was used, for example, by Peters-Wendisch (Microbiology 144, 915 -927 (1998)) I.B.R. to eliminate the pyc gene of C. glutamicum by a deletion.

A deletion, insertion or a base exchange can be incorporated into the alr gene in this manner.

It may furthermore be advantageous for the production of L-glutamic acid, in addition to the attenuation of D-alanine racemase, for one or more of the genes chosen from the group consisting of:

the gap gene which codes for glycerolaldehyde 3-phosphate dehydrogenase (Eikmanns (1992). Journal of Bacteriology 174:6076-6086 I.B.R.),

the eno gene which codes for enolase (DE 19947791.4 I.B.R.),

the gdh gene which codes for glutamate dehydrogenase (Börmann et al., Molecular Microbiology 6, 317-326 (1992) I.B.R.),

the gltA gene which codes for citrate synthase (Eikmanns et al., Microbiology 140, 1817-1828 (1994) I.B.R.),

the pyc gene which codes for pyruvate carboxylase (DE-A-198 31 609 I.B.R.),

to be enhanced, in particular over-expressed.

The term “enhancement” or “enhance” in this connection describes the increase in the intracellular activity of one or more enzymes or proteins in a microorganism which are coded by the corresponding DNA, for example by increasing the number of copies of the gene or genes, using a potent promoter or a gene or allele which codes for a corresponding enzyme or protein with a high activity, and optionally combining these measures. It may furthermore be advantageous for the production of L-glutamic acid, in addition to the attenuation of D-alanine racemase, for the pck gene which codes for phosphoenol pyruvate carboxykinase to be attenuated (DE 199 50 409.1 I.B.R., DSM 13047).

Finally, in addition to the attenuation of D-alanine racemase, it may be advantageous for the production of L-glutamic acid to eliminate undesirable side reactions (Nakayama: “Breeding of Amino Acid Producing Micro-organisms”, in: Overproduction of Microbial Products, Krumphanzl, Sikyta, Vanek (eds.), Academic Press, London, UK, 1982) I.B.R.

The microorganisms prepared according to the invention can be cultured continuously or discontinuously in the batch process (batch culture) or in the fed batch (feed process) or repeated fed batch process (repetitive feed process) for the purpose of glutamic acid production. A summary of known culture methods is described in the textbook by Chmiel (Bioprozesstechnik 1. Einführung in die Bioverfahrenstechnik (Gustav Fischer Verlag, Stuttgart, 1991) I.B.R.) or in the textbook by Storhas (Bioreaktoren und periphere Einrichtungen (Vieweg Verlag, Braunschweig/Wiesbaden, 1994) I.B.R.).

The culture medium to be used must meet the requirements of the particular microorganisms in a suitable manner. Descriptions of culture media for various microorganisms are contained in the handbook “Manual of Methods for General Bacteriology” of the American Society for Bacteriology (Washington D.C., USA, 1981) I.B.R.

Sugars and carbohydrates, such as e.g. glucose, sucrose, lactose, fructose, maltose, molasses, starch and cellulose, oils and fats, such as e. g. soya oil, sunflower oil, groundnut oil and coconut fat, fatty acids, such as e.g. palmitic acid, stearic acid and linoleic acid, alcohols, such as e. g. glycerol and ethanol, and organic acids, such as e. g. acetic acid, can be used as the source of carbon. These substance can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the abovementioned substances. The starting substances mentioned can be added to the culture in the form of a single batch, or can be fed in during the culture in a suitable manner.

Basic compounds, such as sodium hydroxide, potassium hydroxide, ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of L-glutamic acid has formed. This target is usually reached within 10 hours to 160 hours.

A pure culture of the Corynebacterium glutamicum strain ATCC13032::pK18mobalr was deposited on Mar. 23, 2001 at the Deutsche Sammlung für Mikroorganismen und Zellkulturen in accordance with the Budapest Treaty (DSMZ=German Collection of Microorganisms and Cell Cultures, Braunschweig, Germany) as DSM 14195.

The present invention is explained in more detail in the following with the aid of embodiment examples.

The isolation of plasmid DNA from Escherichia coli and all techniques of restriction, Klenow and alkaline phosphatase treatment were carried out by the method of Sambrook et al. (Molecular Cloning. A Laboratory Manual (1989) Cold Spring Harbour Laboratory Press, Cold Spring Harbor, N.Y., USA) I.B.R. Methods for transformation of Escherichia coli are also described in this handbook.

The composition of the usual nutrient media, such as LB or TY medium, can also be found in the handbook by Sambrook et al.

### EXAMPLE 1

Preparation of an Integration Vector for Integration Mutagenesis of the alr Gene

Chromosomal DNA from Corynebacterium glutamicum ATCC 13032 was isolated as described by Tauch et al. (Plasmid 33:168-179, 1995). A DNA fragment which carries an internal region of the alr gene was amplified with the aid of the polymerase chain reaction. The following primers were used for this:

| | |
|---|---|
| 5′-TGG ATT TGG ACA CCG GAG CAG GAT-3′ | (SEQ ID |
| | No.3) |
| 5′-AAG GGC GCG TCG AAA AGC AAT AAT-3′ | (SEQ ID |
| | No.4). |

The primers shown were synthesized by MWG Biotech (Ebersberg, Germany) and the polymerase chain reaction was carried out by the standard PCR method of Innis et al., (PCR protocol. A guide to methods and applications, 1990, Academic Press) I.B.R. The primers allow amplification of a DNA fragment of approx. 306 bp in size, which carries an internal region of the alr gene from Corynebacterium glutamicum ATCC13032. After separation by gel electrophoresis, the PCR fragment was isolated from the agarose gel with the QiaExII Gel Extraction Kit (Product No. 20021, Qiagen, Hilden, Germany).

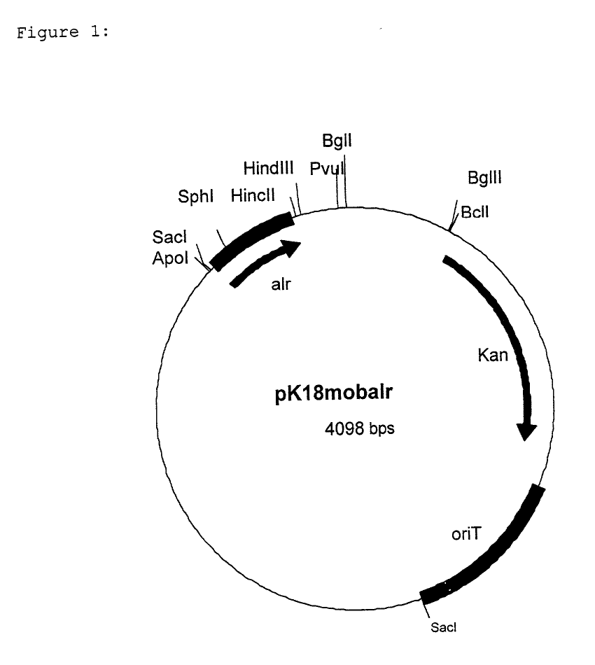

The vector pK18mob (Schäfer et al. 1994, Gene 145:69-73 I.B.R.) was cleaved completely with the restriction endonuclease SmaI and dephosphorylated with shrimp alkaline phosphatase (Roche Diagnostics GmbH, Mannheim, Germany, Product Description SAP, Product No. 1758250). This vector prepared in this way was ligated with the PCR fragment obtained. The ligation batch was transformed in the E. coli strain DH5α (Hanahan, In: DNA cloning. A practical approach. Vol. I. IRL-Press, Oxford, Washington DC, USA I.B.R.). Selection of plasmid-carrying cells was made by plating out the transformation batch on LB agar (Lennox, 1955, Virology, 1:190 I.B.R.) with 100 mg/l ampicillin. After incubation overnight at 37° C., recombinant individual clones were selected. Plasmid DNA was isolated from a transformant with the Qiaprep Spin Miniprep Kit (Product No. 27106, Qiagen, Hilden, Germany) in accordance with the manufacturer's instructions and cleaved with the restriction enzyme SacI to check the plasmid by subsequent agarose gel electrophoresis. The resulting plasmid was called pK18mobalr. The plasmid is shown in FIG. 1.

### EXAMPLE 2

Integration Mutagenesis of the alr Gene in the C. glutamicum Strain ATCC13032

The strain Corynebacterium glutamicum ATCC13032 was subjected to an inactivation mutagenesis as described by Schäfer et al. (1994, Gene 145: 69-73) I.B.R. For this, competent cells of ATCC13032 were prepared as described by van der Rest et al. (Applied Microbiology and Biotechnology (52: 541-545, 1999) I.B.R. These cells were transformed with 200 ng of the vector pK18mobalr and applied to LBHIS agar plates which contained 100 μg/ml D-alanine. Clones in which the inactivation vector was present integrated in the genome were identified with the aid of their kanamycin resistance on LBHIS agar plates containing 15 μg/mL kanamycin (Liebl et al., FEMS Microbiology Letters (1989) 65: 299-304) I.B.R. The integration was checked with the following primer pair:

5′-AAG GGC GCG TCG AAA AGC AAT AAT-3′ (SEQ ID No. 5)

5′-GGA AAC AGC TAT GAC CAT-3′ (SEQ ID No. 6).

A band of about 350 base pairs resulted in the polymerase chain reaction, which indicates integration of the vector pK18mobalr into the alr gene. The resulting strain was called ATCC13032::pK18mobalr and also given the synonymous designation 13032alr⁻.

### EXAMPLE 3

Preparation of L-glutamate with the Aid of the Strain ATCC13032:pK18mobalr

The C. glutamicum strain ATCC13032::pK18mobalr obtained in example 2 was cultured in a nutrient medium suitable for the production of glutamate and the glutamate content in the culture supernatant was determined.

For this, the strain was first incubated on an agar plate with the corresponding antibiotic (brain-heart agar with kanamycin (50 mg/l) and D-alanine (50 mg/l) for 24 hours at 33° C. Starting from this agar plate culture, a preculture was seeded (10 ml medium in a 100 ml conical flask). The medium used for the preculture was the complete medium CgIII (2.5 g/l NaCl, 10 g/l Bacto-Peptone, 10 g/l Bacto-Yeast Extract, pH7.4, 20 g/l glucose (autoclaved separately). Kanamycin (25 mg/l) and D-alanine (50 mg/l) were added to this. The preculture was incubated for 16 hours at 33° C. at 240 rpm on a shaking machine. A main culture was seeded from this preculture such that the initial OD (660 nm) of the main culture was 1. The production medium CgXII (Keilhauer et al. 1993, Journal of Bacteriology 175:5595-5603 I.B.R.) was used for the main culture. 4% glucose and 25 mg/l kanamycin sulfate were added. Culturing is carried out in a volume of 10 ml in a 100 ml conical flask with baffles with the addition of various D-alanine concentrations. Culturing was carried out at 37° C. and 80% atmospheric humidity. After 24 hours, the OD was determined at a measurement wavelength of 660 nm with a Biomek 1000 (Beckmann Instruments GmbH, Munich, Germany). The glutamate concentration formed was determined with an amino acid analyzer from Eppendorf-BioTronik (Hamburg, Germany) by ion exchange chromatography and post-column derivatization with ninhydrin detection. The dry weight (DM) was calculated with the aid of the OD, on the basis that an OD of 40 corresponds to a dry weight of 12 g per litre.

The result of the experiment is shown in Table 1.

TABLE 1

| | D-Alanine | L-Glutamate |
|---|---|---|
| | addition | production |
| Strain | mg 1⁻⁴ | mmol (g DM) ⁻¹ |
| 13032 | 0 | 0.06 |
| 13032alr⁻ | 0 | 1.45 |
| 13032alr⁻ | 10 | 0.68 |

We claim:

1. A method for preparing L-glutamic acid comprising fermenting coryneform bacteria in which the nucleotide sequence which codes for D-alanine racemase (alr) is attenuated.

2. The method according to claim 1, wherein bacteria in which the nucleotide sequence coding for D-alanine racemase (alr) is eliminated are employed.

3. The method according to claim 1, comprising
a) fermenting, in a medium, the L-glutamic acid-producing bacteria in which at least the gene which codes for D-alanine racemase is attenuated.

4. The method according to claim 3, further comprising
b) concentrating the L-glutamic acid in the medium or in cells of the bacteria, and

5. The method according to claim 4, further comprising
c) isolating the L-glutamic acid produced.

6. The method according to claim 1, wherein in said bacteria one or more of the genes chosen from the group consisting of
a. the gap gene which codes for glycerolaldehyde 3-phosphate dehydrogenase,
b. the eno gene which codes for enolase,
c. the gdh gene which codes for glutamate dehydrogenase,
d. the gltA gene which codes for citrate synthase, and
e. the pyc gene which codes for pyruvate carboxylase,
are enhanced.

7. The method according to claim 1, wherein in said bacteria one or more of the genes chosen from the group consisting of
a. the gap gene which codes for glycerolaldehyde 3-phosphate dehydrogenase,
b. the eno gene which codes for enolase,
c. the gdh gene which codes for glutamate dehydrogenase,
d. the gltA gene which codes for citrate synthase, and
e. the pyc gene which codes for pyruvate carboxylase,
are over-expressed.

8. The method as claimed in claim 1, wherein said bacteria are a Corynebacterium glutamicum strain ATCC13032::pK18mobalr, DSM 14195.


SEQUENCE LISTING (6 sequences)

SEQ ID NO: 1 - DNA, 1843 bp, Corynebacterium glutamicum
  CDS (487)..(1572)
agtactgcagccccagcacccatgccgtggcctactacaccgaggcaggctgggttgacggtgacgtttccggagccgagttttacgccgccgagaatctgaatggaggattcgaggtcagaggcgaaacctttgtggtctggcatgaagccattttcggtgtctggggcggcaacagcgatgccccaggacgcgaggtgtcgcaaagtttggtggtagtacttgatggatttcatccagtcgtggccgaaagctacacctgggatgccgtcgccttctgctggggtgtagattttgcccgggatgccggcgtagttcatatcgcctaccagcacgcggtgcggtccgcgcttggacagtttggacaggtgtttgttcagattctcagccacgtgtttaaggatagttgaaagcgtggggcaatactggcactaaccccggcaccaatcgtatttctgtccgcggttggtggcacaatagttcaacatgaacttgctgaccaccaaaattgacctggatgccatcgcccataacacgagggtgcttaaacaaatggcgggtccggcgaagctgatggcggtggtgaaggcgaatgcatataaccatggcgtagagaaggtcgctccggttattgctgctcatggtgcggatgcgtttggtgtggcaactcttgcggaggctatgcagttgcgtgatatcggcatcagccaagaggttttgtgttggatttggacaccggagcaggatttccgcgccgccattgatcgcaatattgatttggctgttatttctcccgcgcatgccaaagccttgatcgaaactgatgcggagcatattcgggtgtccatcaagattgattctgggttgcatcgttcgggtgtggatgagcaggagtgggagggcgtgttcagcgcgttggctgctgccccgcacattgaggtcacgggcatgttcacgcacttggcgtgcgcggatgagccagagaatccggaaactgatcgccaaattattgcttttcgacgcgcccttgcgctcgcccgcaagcacgggcttgagtgcccggtcaaccacgtatgcaactcacctgcattcttgactcgatctgatttacacatggagatggtccgaccgggtttggccttttatgggttggaacccgtggcgggactggagcatggtttgaagccggcgatgacgtgggaggcgaaggtgagcgtcgtaaagcaaattgaagctggacaaggcacttcctatggcctgacctggcgcgctgaggatcgcggctttgtggctgtggtgcctgcgggctatgccgatggcatgccgcggcatgcccaggggaaattctccgtcacgattgatggcctggactatccgcaggttgggcgcgtatgcatggatcagttcgttatttctttgggcgacaatccacacggcgtggaagctggggcgaaggccgtgatattcggtgagaatgggcatgacgcaactgattttgcggagcgtttagacaccattaactatgaggtagtgtgccgaccaaccggccgaactgtccgcgcatatgtttaagtgaatacgtttaaggagcagcaatgaaatctgagtttccggtatccggcacgaggcgttttgagcatgccgcagatacccaaaattttggggaagaattaggcaggcatctagaagctggcgatgtggtgattttggacggcccgctgggtgctggaaaaaccacatttactcaaggtatcgctcgtggattgcaggtgaaggggcgggtgacatcgccgacgtttgtgatcgcgagggaacaccgctcggaaatcggtgggccagatct

SEQ ID NO: 2 - protein, 361 aa, Corynebacterium glutamicum
MNLLTTKIDLDAIAHNTRVLKQMAGPAKLMAVVKANAYNHGVEKVAPVIAAHGADAFGVATLAEAMQLRDIGISQEVLCWIWTPEQDFRAAIDRNIDLAVISPAHAKALIETDAEHIRVSIKIDSGLHRSGVDEQEWEGVFSALAAAPHIEVTGMFTHLACADEPENPETDRQIIAFRRALALARKHGLECPVNHVCNSPAFLTRSDLHMEMVRPGLAFYGLEPVAGLEHGLKPAMTWEAKVSVVKQIEAGQGTSYGLTWRAEDRGFVAVVPAGYADGMPRHAQGKFSVTIDGLDYPQVGRVCMDQFVISLGDNPHGVEAGAKAVIFGENGHDATDFAERLDTINYEVVCRPTGRTVRAYV

SEQ ID NO: 3 - DNA, 24 bp, Corynebacterium glutamicum
tggatttggacaccggagcaggat

SEQ ID NO: 4 - DNA, 24 bp, Corynebacterium glutamicum
aagggcgcgtcgaaaagcaataat

SEQ ID NO: 5 - DNA, 24 bp, Corynebacterium glutamicum
aagggcgcgtcgaaaagcaataat

SEQ ID NO: 6 - DNA, 18 bp, Corynebacterium glutamicum
ggaaacagctatgaccat